(19) United States
(12) Patent Application Publication
(10) Pub. No.: US 2026/0137506 A1
(43) Pub. Date: May 21, 2026

(54) REPLACEMENT AORTIC VALVE ANCHORING DEVICE

(71) Applicant: BOSTON SCIENTIFIC SCIMED, INC., Maple Grove, MN (US)

(72) Inventor: Donal Kyne, Galway (IE)

(73) Assignee: BOSTON SCIENTIFIC SCIMED, INC., MAPLE GROVE, MN (US)

(21) Appl. No.: 19/394,373

(22) Filed: Nov. 19, 2025

Related U.S. Application Data
(60) Provisional application No. 63/722,156, filed on Nov. 19, 2024.

(51) Int. Cl. A61F 2/24

(52) U.S. Cl. CPC A61F 2/2409; A61F 2/2427; A61F 2220/0016; A61F 2230/0006; A61F 2230/0091; A61F 2250/001; A61F 2250/0039

(57) ABSTRACT

A replacement prosthetic heart valve system includes an anchoring ring sized to be deployed within an aortic annulus, and a replacement prosthetic heart valve configured to be deployed within the anchoring ring.

### CROSS REFERENCE TO RELATED APPLICATIONS

This application claims the benefit of priority of U.S. Provisional Application No. 63/722,156 filed Nov. 19, 2024, the entire disclosure of which is hereby incorporated by reference.

### TECHNICAL FIELD

The present disclosure pertains to medical devices, systems, and methods for manufacturing and/or using medical devices and/or systems. More particularly, the present disclosure is directed to replacement prosthetic heart valve assemblies and more particularly to devices for treating aortic insufficiency and aortic regurgitation.

### BACKGROUND

A wide variety of intracorporeal medical devices have been developed for medical use, for example, intravascular use. Some of these devices include guidewires, catheters, replacement prosthetic heart valves, medical device systems, and the like. These devices are manufactured by any one of a variety of different manufacturing methods and may be used according to any one of a variety of methods. Aortic insufficiency (AI) or aortic regurgitation (AR) is a form of valvular heart disease in which the integrity of the aortic valve is compromised and leads to inadequate closure of the valve leaflets allowing blood to leak back into the ventricle. Annuloplasty is one treatment used to prevent paravalvular leaks without replacing the heart valve. Aortic annuloplasty rings are generally sutured into the inflow portion of the valve, beneath the aortic annulus, with the ring being either rigid or flexible. Standard transcatheter aortic valve replacement (TAVR) devices are also used to treat AI/AR but involve challenges including the absence of calcification for valve anchoring, difficulty in valve sizing, and instability during valve deployment due to increased stroke volume. Ventricular embolization may occur when using a standard TAVR device to treat AI/AR due to a lack of calcification for anchoring. Additionally, paravalvular leaks may occur due to incomplete sealing of the replacement valve within the native valve. The TAVR device is generally oversized up to 20% to help in anchoring, but this often has implications for permanent pacemaker implantation (PPI) and patient prosthesis mismatch. The requirement of a permanent pacemaker not only has the drawback of another surgery and medical device implantation with all its negative implications for the patient but also imposes increased cost in the context of such a heart valve replacement therapy. Accordingly, there is a need to avoid or at least reduce the rate of implanted pacemakers in such treatments and to provide alternative medical devices and systems as well as alternative methods for manufacturing and using such medical devices.

### SUMMARY

This disclosure provides design, material, manufacturing method, and use alternatives for medical devices. An example replacement prosthetic heart valve system includes an anchoring ring sized to be deployed within an aortic annulus, the anchoring ring defining a single circumferential loop, and a replacement prosthetic heart valve configured to be deployed within the anchoring ring.

Alternatively or additionally to the embodiment above, the anchoring ring is made of metal.

Alternatively or additionally to any of the embodiments above, the anchoring ring is made of fabric or polymer.

Alternatively or additionally to any of the embodiments above, the replacement prosthetic heart valve is configured to be deployed within the anchoring ring with native valve leaflets positioned between the anchoring ring and the replacement prosthetic heart valve.

Alternatively or additionally to any of the embodiments above, the anchoring ring has a first outer diameter and the replacement prosthetic heart valve has a second outer diameter when expanded, wherein the first outer diameter is smaller than the second outer diameter.

Alternatively or additionally to any of the embodiments above, the first outer diameter is 2 mm (0.079 inches) smaller than the second outer diameter.

Alternatively or additionally to any of the embodiments above, the system further includes at least one fixation member coupled to the anchoring ring for securing the anchoring ring to the aortic annulus, the at least one fixation member selected from the group consisting of hooks, pins, screws, helical members, clasps, or barbs.

Alternatively or additionally to any of the embodiments above, the at least one fixation member includes a plurality of pins, each pin having a sharpened distal end and a flattened proximal head, each pin disposed partially through the anchoring ring.

Alternatively or additionally to any of the embodiments above, the at least one fixation member includes a plurality of barbs extending radially outward from an outer surface of the anchoring ring, the plurality of barbs configured to penetrate surrounding tissue of the aortic annulus.

Alternatively or additionally to any of the embodiments above, the at least one fixation member includes a plurality of helical members distributed around a circumference of the anchoring ring, the plurality of helical members configured to be rotated and advanced into surrounding tissue of the aortic annulus.

Alternatively or additionally to any of the embodiments above, the anchoring ring has an adjustable diameter.

Alternatively or additionally to any of the embodiments above, the adjustable diameter is between 18 mm to 30 mm (0.709 inches to 1.181 inches).

Alternatively or additionally to any of the embodiments above, the system further includes a delivery catheter configured for deploying the anchoring ring.

An example kit for securing a replacement prosthetic heart valve, including an anchoring ring sized to be deployed within an aortic annulus, the anchoring ring defining only a single circumferential loop, a plurality of fixation members configured to secure the anchoring ring to the aortic annulus, a delivery catheter configured to receive the anchoring ring, and a replacement prosthetic heart valve configured to be deployed within the anchoring ring.

Alternatively or additionally to the embodiment above, the replacement prosthetic heart valve is configured to be deployed within the anchoring ring with native valve leaflets positioned between the anchoring ring and the replacement prosthetic heart valve.

Alternatively or additionally to any of the embodiments above, the delivery catheter is configured to receive the anchoring ring with at least one of the plurality of fixation members at least partially disposed through the anchoring ring while the anchoring ring is inside the delivery catheter.

An example method of securing a replacement prosthetic heart valve in an aortic annulus includes delivering an anchoring ring to a base of the aortic annulus, securing the anchoring ring to the aortic annulus, and implanting the replacement prosthetic heart valve within the anchoring ring.

Alternatively or additionally to any of the embodiments above, securing the anchoring ring includes attaching the anchoring ring to the aortic annulus using a plurality of fixation members.

Alternatively or additionally to any of the embodiments above, delivering the anchoring ring includes inserting a delivery catheter into the base of the aortic annulus and advancing the anchoring ring through the delivery catheter into the aortic annulus.

Alternatively or additionally to any of the embodiments above, implanting the replacement prosthetic heart valve includes inserting the replacement prosthetic heart valve within native aortic valve leaflets and sandwiching the native aortic valve leaflets between the replacement prosthetic heart valve and the anchoring ring.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE FIGURES

The disclosure may be more completely understood in consideration of the following description in connection with the accompanying drawings, in which:

FIG. 1 illustrates the position of an example anchoring ring within a patient's native aortic valve;

FIGS. 2A-2F illustrate the deployment of an example anchoring ring within the native aortic valve and positioning of a replacement prosthetic heart valve within the anchoring ring and aortic valve;

FIG. 3 is an illustration of the forces from the anchoring ring and replacement prosthetic heart valve on a native aortic valve leaflet;

FIGS. 4A-4C illustrate different fixation members on the anchoring ring; and

FIGS. 5A-5C illustrate another method of deploying an example anchoring ring within the native aortic valve and positioning a replacement prosthetic heart valve within the anchoring ring and aortic valve.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DESCRIPTION

The following description should be read with reference to the drawings, which are not necessarily to scale, wherein like reference numerals indicate like elements throughout the several views. The detailed description and drawings are intended to illustrate example embodiments of the disclosure but not limit the disclosure. Those skilled in the art will recognize that the various elements described and/or shown may be arranged in various combinations and configurations without departing from the scope of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. For example, failure of the aortic valve or the mitral valve can have a serious effect on a human and could lead to a serious health condition and/or death. Treatment of defective heart valves poses challenges in that the treatment often requires the repair or outright replacement of the defective heart valve. Disclosed herein is an apparatus, system, and/or method that may be used in a portion of the cardiovascular system in order to diagnose, treat, and/or repair the system. In some embodiments, the apparatus, system, and/or method disclosed herein may be used before and/or during a procedure to diagnose, treat, and/or repair a defective heart valve (e.g., the aortic valve, the mitral valve, etc.). In addition, a replacement prosthetic heart valve implant may be delivered percutaneously and thus may be much less invasive to the patient. The apparatus, system, and/or method disclosed herein may also provide other desirable features and/or benefits as described below.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the leaflet”, “the strut”, or other features may be equally referred to all instances and quantities beyond one of said feature unless clearly stated to the contrary. As such, it will be understood that the following discussion may apply equally to any and/or all of the components for which there are more than one within the replacement prosthetic heart valve implant and/or the apparatus unless explicitly stated to the contrary.

Additionally, it should be noted that in any given figure, some features may not be shown, or may be shown schematically, for clarity and/or simplicity. Additional details regarding some components and/or method steps may be illustrated in other figures in greater detail. The systems, devices, and/or methods disclosed herein may provide a number of desirable features and benefits as described in more detail below. For the purpose of this disclosure, the discussion below is directed toward the treatment of a native aortic valve and will be so described in the interest of brevity. This, however, is not intended to be limiting as the skilled person will recognize that the following discussion may also apply to a mitral valve or another heart valve with no or minimal changes to the structure and/or scope of the disclosure. Similarly, the medical devices disclosed herein may have applications and uses in other portions of a patient's anatomy, such as but not limited to, arteries, veins, and/or other body lumens.

Prosthetic or replacement prosthetic heart valves are medical devices used to replace damaged or diseased natural heart valves. These valves typically consist of an expandable frame and a valve assembly. Various replacement prosthetic heart valves for aortic, mitral, and tricuspid heart valves are currently available, including TAVR devices. Example replacement prosthetic heart valves are described in U.S. Pat. Nos. 10,206,774, 10,285,809, 10,357,359, 10,376,359, 10,420,658, 10,426,608, 10,426,609, 10,980,636, and 11,957,573, the disclosures of which are incorporated herein by reference.

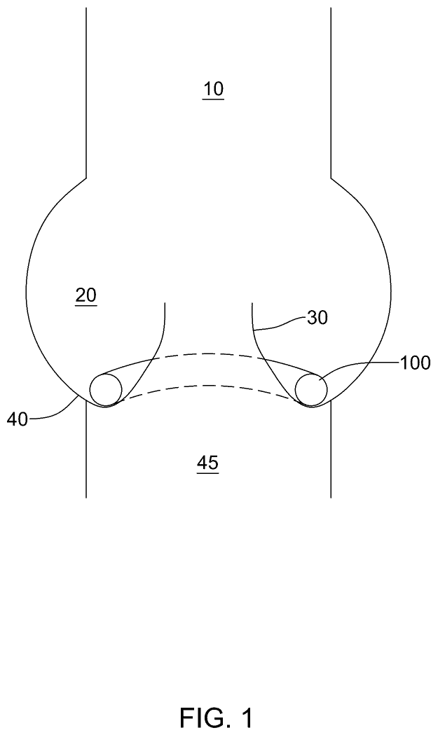

Instead of developing a completely new replacement prosthetic heart valve, a system has been developed for use with a replacement prosthetic heart valve such as a TAVR device for AI/AR disease states. The system may include an anchoring ring and a replacement prosthetic heart valve. FIG. 1 illustrates the position of a deployed anchoring ring 100 within a patient's aorta 10. The anchoring ring 100 may be sized to be deployed into an aortic sinus 20 and positioned at the base of an aortic annulus 40, behind native valve leaflets 30. No part, component, or aspect of the anchoring ring 100 is disposed in a left ventricle 45.

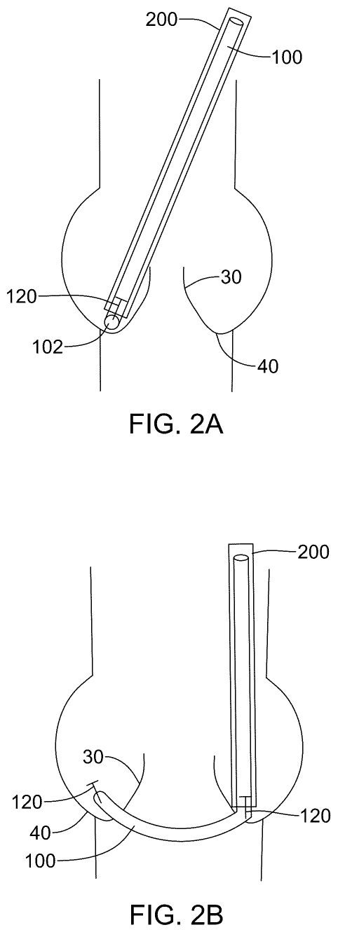

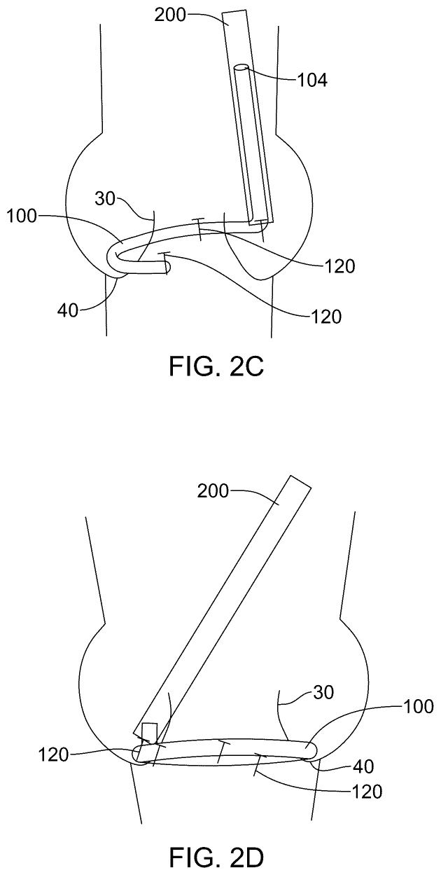

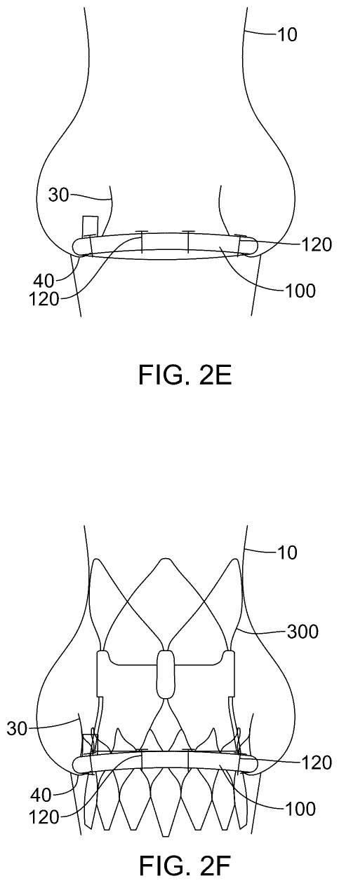

FIGS. 2A-2F illustrate a method of securing a replacement prosthetic heart valve 300, such as a TAVR device, in the patient's aortic valve with the anchoring ring 100. In FIG. 2A, a delivery catheter 200 is inserted through the aorta and a distal end of the delivery catheter 200 is positioned within the sinus adjacent the base of the annulus, outside of the native valve leaflets. The anchoring ring 100 is slidably disposed within the delivery catheter 200. In some embodiments, the anchoring ring 100 may be deployed by pushing the anchoring ring 100 out of the delivery catheter 200 with a pushing member (not shown). In some embodiments, a plurality of fixation members 120 may be coupled to the anchoring ring 100 for securing the anchoring ring directly to the aortic annulus 40, behind the native valve leaflets 30. The fixation members 120 may be coupled to the anchoring ring 100 before the anchoring ring 100 is inserted into the delivery catheter 200. In some embodiments, the fixation members 120 may be hooks, pins, screws, clasps, barbs, or a combination thereof. When the fixation members 120 include pins or screws, the pins or screws may be disposed partially in the anchoring ring 100 during deployment. As shown in FIG. 2A, a first pin 120 is shown partially inserted through the anchoring ring 100 while the anchoring ring 100 is disposed within the delivery catheter 200. Once the anchoring ring 100 is delivered into the annulus, the first pin 120 and all other pins are pushed or secured into the tissue of the annulus. In other embodiments, the anchoring ring 100 may be fully deployed into the base of the annulus and then a plurality of fixation members 120 may be inserted into the anchoring ring 100 to secure it within the annulus.

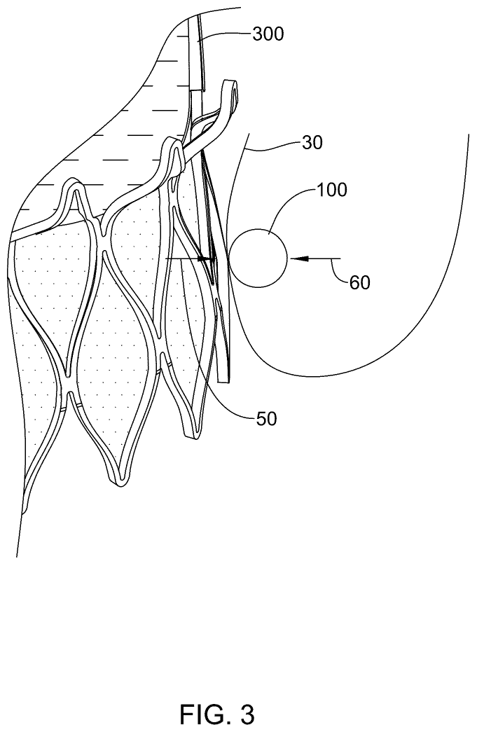

The delivery catheter 200 may be moved around the annulus to deliver the entire anchoring ring 100 at the base of the annulus, as shown in FIGS. 2B-2D. The anchoring ring 100 may be deployed as a tubular structure formable into any shape. In some embodiments, the delivery catheter 200 may be moved to create a circular shape as the delivery catheter 200 is moved around the annulus, delivering the anchoring ring 100, as shown in FIGS. 2B-2D. As each fixation member 120 exits the delivery catheter 200, the fixation member 120 may be secured to the annulus. In some embodiments, a separate device may be inserted into the annulus to push, pin, screw, or otherwise secure the fixation member 120 into the tissue at the base of the annulus. In some embodiments, the anchoring ring 100 may be made of a shape memory material, either metal or polymer. A shape memory anchoring ring 100 may form a ring shape as it is deployed from the delivery catheter 200. The anchoring ring 100 may be sized to extend circumferentially around the annulus to form a single ring, as shown in FIG. 2E. The entirety of the anchoring ring 100 may be disposed within the annulus, behind the native valve leaflets 30. In some embodiments, the distal end 102 of the anchoring ring 100 and the proximal end 104 of the anchoring ring 100 may overlap slightly or abut one another once the anchoring ring 100 has been fully deployed within the annulus. In other embodiments, the distal end 102 of the anchoring ring 100 may be spaced apart from the proximal end 104 when fully deployed. However, the anchoring ring 100 defines a single circumferential ring, and does not form a coil. Once the anchoring ring 100 is secured to the base of the annulus, the delivery catheter may be withdrawn, as shown in FIG. 2E. A TAVR device or other replacement prosthetic heart valve 300 may then be inserted into the native heart valve, inside the native valve leaflets 30, and expanded, as shown in FIG. 2F. When the replacement prosthetic heart valve 300 is expanded, the native valve leaflets 30 may be sandwiched or held between the anchoring ring 100 and the replacement prosthetic heart valve 300, with no direct contact between the anchoring ring 100 and the replacement prosthetic heart valve 300, as shown in FIG. 3. The replacement prosthetic heart valve 300 may be sized according to the size of the native heart valve, without being oversized. In some embodiments, the circumference of the anchoring ring 100 may be smaller than the circumference of the replacement prosthetic heart valve 300 to ensure anchoring via a tight fit between the anchoring ring 100 on one side of the native valve leaflets 30 and the replacement prosthetic heart valve 300 on the other side of the native valve leaflets 30. For example, the anchoring ring 100 may have a circumference that is 1 millimeter (mm) to 4 mm (0.039 inches to 0.157 inches) smaller than the outer circumference of the replacement prosthetic heart valve 300. In one embodiment, the anchoring ring 100 may have a circumference that is 2 mm (0.079 inches) smaller than the outer circumference of the replacement prosthetic heart valve 300. This difference in circumference may help secure the replacement prosthetic heart valve 300 within the native heart valve, even without calcification on the native leaflets, and without using an oversized replacement prosthetic heart valve. The anchoring ring 100 may prevent paravalvular leakage due to the replacement prosthetic heart valve 300 not sealing completely to the native valve leaflets. The anchoring ring 100 provides a radially inward compressive force (arrow 60) on the native valve leaflets 30 while the replacement prosthetic heart valve 300 provides a radially outward compressive force (arrow 50) on the native valve leaflets 30. See FIG. 3. These opposing forces may provide a stronger securement of the replacement prosthetic heart valve 300 to the native valve than the outward force of the replacement prosthetic heart valve alone.

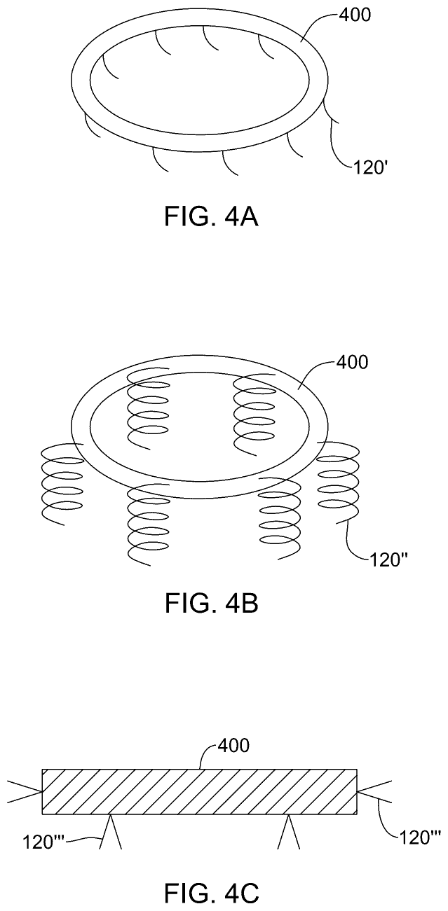

Various types of fixation members 120 may be used to secure the anchoring ring 100 to the annulus. The fixation members 120 illustrated in FIGS. 2A-2F are pins with simple shafts having a sharpened distal tip for penetrating tissue and a flattened enlarged proximal head for receiving an impact force to push the pin into tissue. In other embodiments, the fixation members 120 may be hooks or barbs 120′ as shown in FIG. 4A, screws or helical members 120″, as shown in FIG. 4B, or clasps 120″′ extending radially outward as shown in FIG. 4C. When the fixation members 120 are pins 120 or screws or helical members 120″, they may be delivered partially embedded within the anchoring ring 100 and extended downward such that they may be fully advanced into the bottom of the annulus using a tool delivered through the delivery catheter 200 or through another shaft. When the fixation members 120 are a plurality of hooks or barbs 120′, the hooks or barbs 120′ may extend radially outward from an outer surface of the anchoring ring 100, 400, as shown in FIG. 4A, with the hooks or barbs 120′ configured to penetrate surrounding tissue of the aortic annulus when deployed. When the fixation members 120 are a plurality of screws or helical members 120″, they may be distributed around the circumference of the anchoring ring 100, 400 and be configured to be rotated and advanced into the surrounding tissue of the aortic annulus when deployed. When the plurality of fixation members 120 are a plurality of clasps 120″′, they may be disposed on an outer side surface and/or bottom surface of the anchoring ring 100, 400, as shown in FIG. 4C such that when the anchoring ring 100, 400 is positioned in the bottom of the annulus, the clasps 120″′ may grasp the sidewalls and/or bottom surface of the sinus. The clasps 120″′ may be made from a shape memory material that has a heat set closed configuration. The clasps 120″′ may be forced open within the delivery catheter, and close to pinch tissue and secure the anchoring ring 100, 400 upon deployment out of the delivery catheter 200.

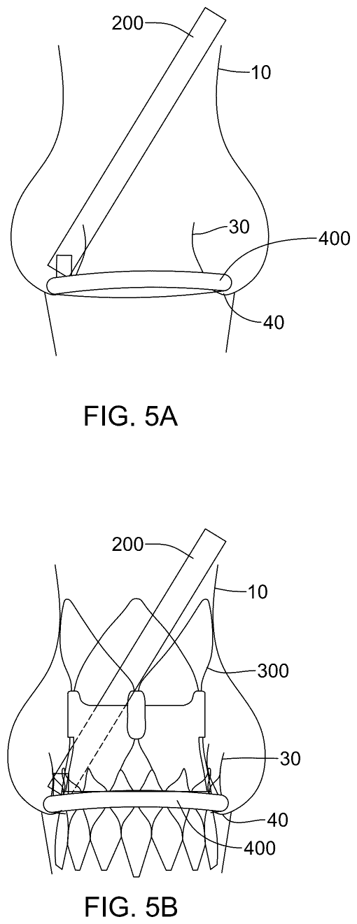

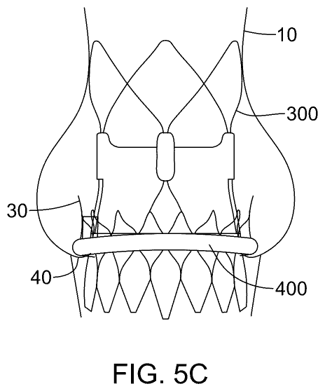

In another embodiment, an anchoring ring 400 without any fixation members may be delivered through the delivery catheter 200 and positioned within the base of the annulus. See FIG. 5A. In some embodiments, the anchoring ring 400 may be held in position with the delivery catheter during insertion of a replacement prosthetic heart valve 300 within the native heart valve, as shown in FIG. 5B. Once the replacement prosthetic heart valve 300 has been expanded within the native valve, the delivery catheter may be withdrawn, with the anchoring ring 400 and the replacement prosthetic heart valve 300 applying compressive force on opposite sides of the native valve leaflets, as shown in FIG. 5C. As described above with regard to FIG. 3, the anchoring ring 400 provides a radially inward compressive force on the native valve leaflets 30 while the replacement prosthetic heart valve 300 provides a radially outward compressive force on the native valve leaflets 30. In this embodiment, the opposing forces of the anchoring ring 400 and the replacement prosthetic heart valve 300 are the only means of attaching the anchoring ring 400 within the annulus. In other embodiments, the delivery catheter 200 may be withdrawn before the replacement prosthetic heart valve 300 is inserted.

In all of the embodiments described above, the anchoring ring 100, 400 may be made of metal, such as nitinol, polymer, or fabric. The metal or polymer may be a shape memory material. In some embodiments, the fabric may be polyester, such as Dacron®. In other embodiments, the anchoring ring 100, 400 may be made of metal or polymer and covered with fabric. A portion or the entire outer surface of the anchoring ring 100, 400 may be roughened to provide friction with the annular surface and help hold the anchoring ring 100, 400 in place during delivery. The thickness of the anchoring ring 100, 400 may be between 1.5 mm and 5 mm (0.059 inches and 0.197 inches). In some embodiments, the anchoring ring 100, 400 may have a thickness of between 2 mm and 4 mm (0.079 inches and 0.157 inches). When the anchoring ring 100, 400 is cylindrical, the thickness may be measured as the transverse diameter.

In some embodiments a kit or assembly with a plurality of sizes of replacement prosthetic heart valves and a plurality of sizes of anchoring rings 100, 400 may be provided. For example, TAVR devices having an expanded diameter of 20 mm, 25 mm, 27 mm, 29 mm, and 31 mm (0.787, 0.984, 1.063, 1.142, and 1.220 inches) may be provided with anchoring rings 100, 400 having a diameter of 18 mm, 23 mm, 25 mm, 27 mm, and 29 mm (0.709, 0.906, 0.984, 1.063, and 1.142 inches). The various replacement prosthetic heart valves may be deployed with an anchoring ring 100, 400 having an outer diameter that is 2 mm (0.079 inches) smaller than the outer diameter of the replacement prosthetic heart valve to achieve a desired level of compressive forces on the native valve leaflets.

In some embodiments, the anchoring ring 100, 400 may be expandable to have an adjustable diameter. The diameter of the anchoring ring 100, 400 may be adjustable between about 15 mm to 30 mm (0.591 to 1.181 inches). An anchoring ring 100, 400 with an adjustable diameter may be used as an alternative to providing a selection of anchoring rings 100, 400 sized to be used with the standard replacement prosthetic heart valves. In some embodiments an adjustable anchoring ring 100, 400 may be made of an expandable polymer such as a shape memory polymer.

In a further embodiment, an expandable anchoring ring 100, 400 may be expanded by a balloon as part of or alternatively to a pre-dilation procedure. For example, the anchoring ring 100, 400 may be delivered in a contracted configuration, either through the delivery catheter 200 or on a balloon. If delivered on a balloon, the balloon may be inserted into the native valve with the anchoring ring 100, 400 positioned just downstream of the native valve leaflets. Upon expansion of the balloon, the anchoring ring 100, 400 may be expanded to a size configured to fit into the annulus. The anchoring ring 100, 400 may be deployed into the base of the annulus and the balloon may be deflated and withdrawn. A plurality of fixation members 120 may be disposed within the anchoring ring 100 and attached to the annulus as described above. Alternatively, the anchoring ring 400 may be positioned within the base of the annulus without any securing means, other than the replacement prosthetic heart valve delivered into the native heart valve and expanded to sandwich the native valve leaflets between the anchoring ring 400 and the replacement prosthetic heart valve 300.

The system of the anchoring ring 100, 400 and the replacement prosthetic heart valve 300 may protect the patient from conduction disturbances. The atrioventricular (AV) node is a nerve in the heart that conducts electrical impulses from the atria to the ventricles, controlling the heart rate. When an oversized TAVR device is used to treat AI/AR, the device may impede function of the AV node, resulting in the need for a permanent pacemaker. Because the system of the anchoring ring 100, 400 and a replacement prosthetic heart valve 300 eliminates the need for the replacement prosthetic heart valve to be oversized, the AV node may be protected.

The materials that can be used for the various components of the medical device system and the various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the anchoring ring 100, 400, the replacement prosthetic heart valve 300, delivery catheter 200, fixation members 120, and/or elements or components thereof.

In some embodiments, the additional system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyisobutylene (PIB), polyisobutylene polyurethane (PIBU), polyurethane silicone copolymers (for example, Elast-Eon® from AorTech Biomaterials or ChronoSil® from AdvanSource Biomaterials), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof, may be made of a material that does not substantially distort the image and create substantial artifacts (i.e., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni-Co-Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A replacement prosthetic heart valve system comprising:
an anchoring ring sized to be deployed within an aortic annulus, the anchoring ring defining a single circumferential loop; and
a replacement prosthetic heart valve configured to be deployed within the anchoring ring.

2. The system of claim 1, wherein the anchoring ring is made of metal.

3. The system of claim 1, wherein the anchoring ring is made of fabric or polymer.

4. The system of claim 1, wherein the replacement prosthetic heart valve is configured to be deployed within the anchoring ring with native valve leaflets positioned between the anchoring ring and the replacement prosthetic heart valve.

5. The system of claim 1, wherein the anchoring ring has a first outer diameter and the replacement prosthetic heart valve has a second outer diameter when expanded, wherein the first outer diameter is smaller than the second outer diameter.

6. The system of claim 5, wherein the first outer diameter is 2 mm (0.079 inches) smaller than the second outer diameter.

7. The system of claim 1, further comprising at least one fixation member coupled to the anchoring ring for securing the anchoring ring to the aortic annulus, the at least one fixation member selected from the group consisting of hooks, pins, screws, helical members, clasps, or barbs.

8. The system of claim 7, wherein the at least one fixation member includes a plurality of pins, each pin having a sharpened distal end and a flattened proximal head, each pin disposed partially through the anchoring ring.

9. The system of claim 7, wherein the at least one fixation member includes a plurality of barbs extending radially outward from an outer surface of the anchoring ring, the plurality of barbs configured to penetrate surrounding tissue of the aortic annulus.

10. The system of claim 7, wherein the at least one fixation member includes a plurality of helical members distributed around a circumference of the anchoring ring, the plurality of helical members configured to be rotated and advanced into surrounding tissue of the aortic annulus.

11. The system of claim 1, wherein the anchoring ring has an adjustable diameter.

12. The system of claim 11, wherein the adjustable diameter is between 18 mm to 30 mm (0.709 inches to 1.181 inches).

13. The system of claim 1, further comprising a delivery catheter configured for deploying the anchoring ring.

14. A kit for securing a replacement prosthetic heart valve, comprising:
an anchoring ring sized to be deployed within an aortic annulus, the anchoring ring defining only a single circumferential loop;
a plurality of fixation members configured to secure the anchoring ring to the aortic annulus;
a delivery catheter configured to receive the anchoring ring; and
a replacement prosthetic heart valve configured to be deployed within the anchoring ring.

15. The kit of claim 14, wherein the replacement prosthetic heart valve is configured to be deployed within the anchoring ring with native valve leaflets positioned between the anchoring ring and the replacement prosthetic heart valve.

16. The kit of claim 14, wherein the delivery catheter is configured to receive the anchoring ring with at least one of the plurality of fixation members at least partially disposed through the anchoring ring while the anchoring ring is inside the delivery catheter.

17. A method of securing a replacement prosthetic heart valve in an aortic annulus, the method comprising:
delivering an anchoring ring to a base of the aortic annulus;
securing the anchoring ring to the aortic annulus; and
implanting the replacement prosthetic heart valve within the anchoring ring.

18. The method of claim 17, wherein securing the anchoring ring includes attaching the anchoring ring to the aortic annulus using a plurality of fixation members.

19. The method of claim 17, wherein delivering the anchoring ring includes inserting a delivery catheter into the base of the aortic annulus and advancing the anchoring ring through the delivery catheter into the aortic annulus.

20. The method of claim 17, wherein implanting the replacement prosthetic heart valve includes inserting the replacement prosthetic heart valve within native aortic valve leaflets and sandwiching the native aortic valve leaflets between the replacement prosthetic heart valve and the anchoring ring.
